(19) United States
(12) Patent Application Publication
(10) Pub. No.: US 2026/0157657 A1
(43) Pub. Date: Jun. 11, 2026

(54) Methods and Systems for Determining the Likelihood of Mobility Disorders

(71) Applicant: IDEXX Laboratories, inc., Westbrook, ME (US)

(72) Inventors: Timothy Gardner, Portland, ME (US); Evan Peck, Mechanicsburg, PA (US)

(73) Assignee: IDEXX Laboratories, inc., Westbrook, ME (US)

(21) Appl. No.: 19/301,405

(22) Filed: Aug. 15, 2025

Related U.S. Application Data
(60) Provisional application No. 63/728,816, filed on Dec. 6, 2024.

(51) Int. Cl. A61B 5/11; A61B 5/00; G06N 20/00

(52) U.S. Cl. CPC A61B 5/112; A61B 5/1127; A61B 5/6801; A61B 5/7267; G06N 20/00; A61B 2503/40; A61B 2562/0219

(57) ABSTRACT

A computer implemented method for identifying a condition is disclosed. The method includes receiving movement data associated with the movement of a subject over a configurable period of time. The method also includes associating the received movement data with a profile of the subject, the profile comprising anatomical data of the subject. The method additionally includes applying a machine learning algorithm to the received movement data and the profile. The method further includes determining, via the machine learning algorithm and based at least in part on the received movement data and the anatomical data of the subject, a likelihood of a mobility disorder.

### CROSS-REFERENCE TO RELATED APPLICATIONS

This application claims the benefit of co-pending U.S. Provisional Patent Application Ser. No. 63/728,816, filed Dec. 6, 2024, which is hereby incorporated by reference in its entirety.

### FIELD OF THE DISCLOSURE

The present disclosure involves models and systems for veterinary applications. More particularly, the present disclosure relates to machine learning models for analyzing and determining the risk of mobility disorders in animal subjects.

### BACKGROUND

Mobility disorders, such as osteoarthritis and hip dysplasia, are common among animals, such as dogs and cats. Osteoarthritis is a degenerative joint disease that causes pain, stiffness, and swelling the joints. Symptoms of osteoarthritis can include lameness, reduced activity, stiffness, limping, changes in behavior, and reduced activity. Hip dysplasia is a condition that causes abnormal hip joint development. It can affect one or more hip joints and can range from a mild abnormality to a complete dislocation. Hip dysplasia can lead to bone degeneration, pain, and reduced mobility.

Typically, diagnoses of these disorders in animals involves a screening during a clinical veterinary visit. More particularly, veterinarians generally rely on abnormalities in a physical exam to recognize the possibility or increased likelihood of mobility disorders. In some instances, additional diagnostic, laboratory testing, and/or imaging (e.g., X-Rays, Computed Tomography (CT) scan, and/or magnetic resonance imaging (MRI)), may be required to definitively diagnose a disorder.

Early diagnosis and treatment of these mobility disorders can help manage the pain and progression of the disorder. However, the lack of clinical signs and the absence of adequate screening can make it difficult to diagnose mobility disorders at an early stage, where interventions and therapies are most effective. As it is easy for a veterinarian to miss the signs of and/or recognize an increased likelihood of developing mobility disorders, especially in the early stages, there exists a need for a new approach that utilizes machine learning to assess the risk for mobility disorders at an early stage.

### SUMMARY

In an example, a computer implemented method for identifying a condition, the method including receiving movement data associated with movement of a subject over a configurable period of time. The method also includes associating the received movement data with a profile of the subject, the profile comprising anatomical data of the subject. The method additionally includes applying a machine learning algorithm to the received movement data and the profile. The method further includes determining, via the machine learning algorithm and based at least in part on the received movement data and the anatomical data of the subject, a likelihood of a mobility disorder.

In another example, a non-transitory computer-readable medium, having stored thereon program instructions that, upon execution by a processor, cause performance of a set of operations including receiving movement data associated with movement of a subject over a configurable period of time. The set of operations also includes associating the received movement data with a profile of the subject, the profile comprising anatomical data of the subject. The set of operations additionally includes applying a machine learning algorithm to the received movement data and the profile. The set of operations further includes determining, via the machine learning algorithm and based at least in part on the received movement data and the anatomical data of the subject, a likelihood of a mobility disorder.

In another example, a computing system includes a processor and a non-transitory computer-readable medium, having stored thereon program instructions that, upon execution by the processor, cause performance of a set of operations. The set of operations includes receiving movement data associated with movement of a subject over a configurable period of time. The set of operations also includes associating the received movement data with a profile of the subject, the profile comprising anatomical data of the subject. The set of operations additionally includes applying a machine learning algorithm to the received movement data and the profile. The set of operations further includes determining, via the machine learning algorithm and based at least in part on the received movement data and the anatomical data of the subject, a likelihood of a mobility disorder.

The features, functions, and advantages that have been discussed can be achieved independently in various examples or may be combined in yet other examples. Further details of the examples can be seen with reference to the following description and drawings.

### BRIEF DESCRIPTION OF THE FIGURES

The above, as well as additional features will be better understood through the following illustrative and non-limiting detailed description of example embodiments, with reference to the appended drawings.

FIG. 1 illustrates a simplified block diagram of an example computing device, according to an example embodiment.

FIG. 2 illustrates a measurement device configured for use with an assessment platform and a mobile computing device, according to an example embodiment.

FIG. 3 illustrates a method, according to an example embodiment.

All the figures are schematic, not necessarily to scale, and generally only show parts which are necessary to elucidate example embodiments, wherein other parts may be omitted or merely suggested.

### DETAILED DESCRIPTION

Example embodiments will now be described more fully hereinafter with reference to the accompanying drawings. That which is encompassed by the claims may, however, be embodied in many different forms and should not be construed as limited to the embodiments set forth herein; rather, these embodiments are provided by way of example. Furthermore, like numbers refer to the same or similar elements or components throughout.

Within examples, the present disclosure is directed to methods and systems for determining the likelihood of a mobility disorder in a subject, such as a dog or a cat. For instance, systems and methods of the present disclosure incorporate a machine learning algorithm to determine the likelihood of a mobility disorder analyzing movement data of a subject in view of information about the subject (e.g., breed, sex, weight, etc.). This can help veterinarians diagnose mobility disorders earlier and more accurately. This can also help pet owners intervene to help manage the pain and progression of a disorder.

Historically, veterinarians rely on abnormalities in a physical exam to recognize the possibility or increased likelihood of mobility disorders. Thus, veterinarians are typically rely on the limited information gathered during a clinical visit to diagnose a subject. In example implementations of the present disclosure, the movement data may be collected by devices in “at-home” settings (i.e., not during a clinical veterinary visit) over periods of time. For instance, the subject may have a wearable device attached to their limb (e.g., a boot) or their neck (e.g., a collar) over a period of time. The wearable device can collect data related to the pace, location, and/or movement patterns of the subject, for example. In another example, movement data can include one or more pictures or videos of the subject walking and/or running. Accordingly, movement data can be collected in various settings and over longer periods of time, as it is not limited to observations and/or information collected during a clinical veterinary visit.

The collected movement data is transmitted to an assessment platform and input into a machine learning algorithm for analysis. For example, the machine learning algorithm may determine an abnormality (e.g., a limp) in the subject's gait. In examples, the machine learning algorithm can assess and determine the severity of an abnormality. For instance, the machine learning algorithm may distinguish a slight limp from a more severe limp. Alternatively, the machine learning algorithm may determine that the subject has a normal gait.

The machine learning algorithm can also access a profile of the subject, which includes anatomical data about the subject. For instance, anatomical data can include information such as breed, sex, and/or weight of the subject. Anatomical data may additionally or alternatively include information related to the subject's medical records. The subject's medical records may include, but is not limited to, demographic information, vital signs at each clinical visit, diagnoses, medications, treatment plans, progress notes, subject problems, vaccine history, test results, and imaging data, such as radiographs.

The machine learning algorithm then determines the likelihood of mobility disorder based both on the received movement data and the profile of the subject. For instance, the machine learning algorithm may determine there is high likelihood of a mobility disorder if the movement data indicates a severe abnormality in subject's the weight distribution between limbs and information in the subject's profile indicates a higher likelihood of a mobility disorder (e.g., the subject is overweight for the particular breed). In another example, the machine learning algorithm may detect a slight limp in the subject's movement data, however information in the subject's profile does not indicate any increased risk factors. In this case, the machine learning algorithm may determine that there is a lower likelihood of a mobility disorder. Many example implementations and scenarios are possible.

In examples, the assessment platform may transmit instructions that cause a computing device, such as a smartphone associated with the subject, to display one or more graphical indications of the determined likelihood of the mobility disorder. Additionally or alternatively, the assessment platform may update the profile of the subject with the determination.

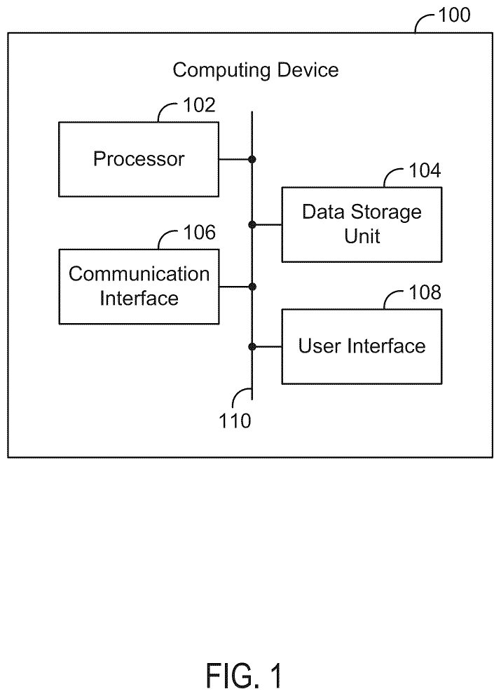

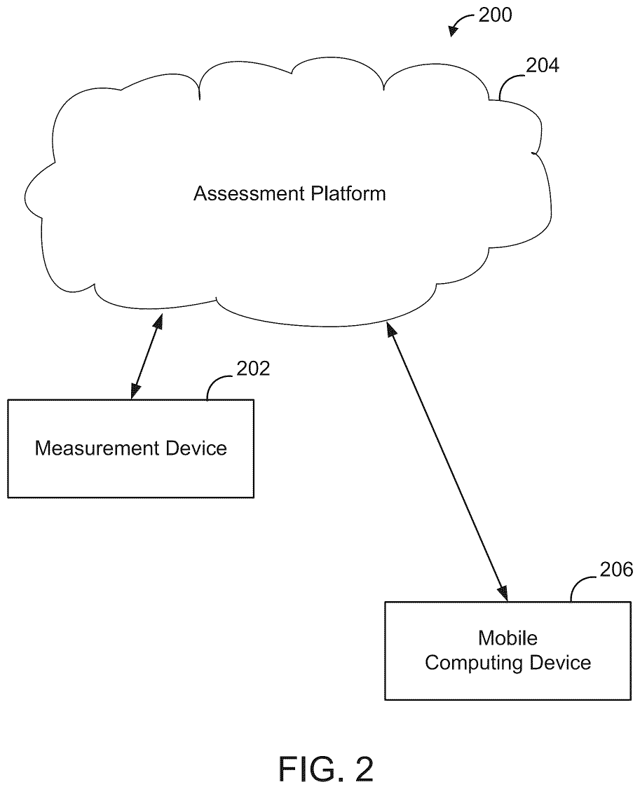

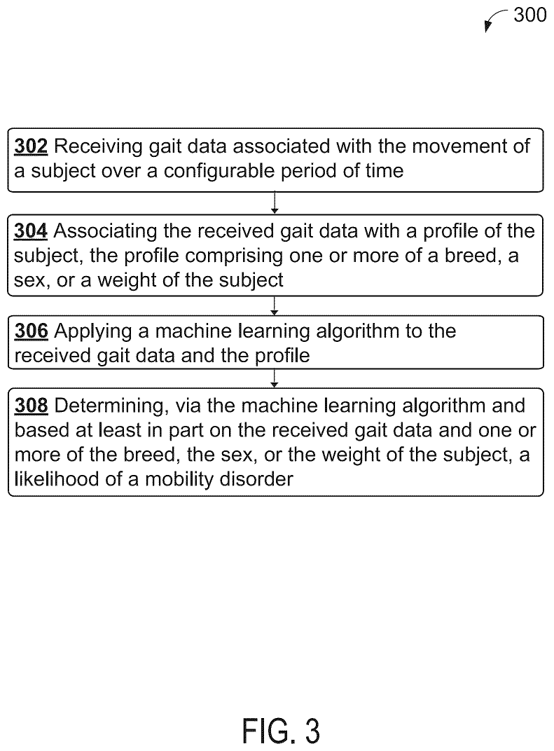

Referring now to the figures, FIG. 1 is a simplified block diagram of an example computing device 100 of a system (e.g., that can be utilized with devices and methods illustrated in FIGS. 2-3, described in further detail below). Computing device 100 can perform various acts and/or functions, such as those described in this disclosure. Computing device 100 can include various components, such as processor 102, data storage unit 104, communication interface 106, and/or user interface 108. These components can be connected to each other (or to another device, system, or other entity) via connection mechanism 110.

Processor 102 can include a general-purpose processor (e.g., a microprocessor and/or a central processing unit (CPU)) and/or a special-purpose processor (e.g., a digital signal processor (DSP) and/or a graphics processing unit (GPU)).

Data storage unit 104 can include one or more volatile, non-volatile, removable, and/or non-removable storage components, such as magnetic, optical, or flash storage, and/or can be integrated in whole or in part with processor 102. Further, data storage unit 104 can take the form of a non-transitory computer-readable storage medium, having stored thereon program instructions (e.g., compiled or non-compiled program logic and/or machine code) that, when executed by processor 102, cause computing device 100 to perform one or more acts and/or functions, such as those described in this disclosure. As such, computing device 100 can be configured to perform one or more acts and/or functions, such as those described in this disclosure. Such program instructions can define and/or be part of a discrete software application. In some instances, computing device 100 can execute program instructions in response to receiving an input, such as from communication interface 106 and/or user interface 108. Data storage unit 104 can also store other types of data, such as those types described in this disclosure.

Communication interface 106 can allow computing device 100 to connect to and/or communicate with another other entity according to one or more protocols. In one example, communication interface 106 can be a wired interface, such as an Ethernet interface or a high-definition serial-digital-interface (HD-SDI). In another example, communication interface 106 can be a wireless interface, such as a cellular or WI FI interface. In this disclosure, a connection can be a direct connection or an indirect connection, the latter being a connection that passes through and/or traverses one or more entities, such as a router, switcher, or other network device. Likewise, in this disclosure, a transmission can be a direct transmission or an indirect transmission.

User interface 108 can facilitate interaction between computing device 100 and a user of computing device 100, if applicable. As such, user interface 108 can include input components such as a keyboard, a keypad, a mouse, a touch sensitive panel, a microphone, a camera, and/or a movement sensor, all of which can be used to obtain data indicative of an environment of computing device 100, and/or output components such as a display device (which, for example, can be combined with a touch sensitive panel), a sound speaker, and/or a haptic feedback system. More generally, user interface 108 can include hardware and/or software components that facilitate interaction between computing device 100 and the user of the computing device 100.

Computing device 100 can take various forms, such as a workstation terminal, a desktop computer, a laptop, a tablet, a mobile phone, or a controller.

Now referring to FIG. 2, a computing system 200 configured for use with a measurement device 202 and a mobile computing device 206, according to an example embodiment. The measurement device 202 and the mobile computing device 206 are both communicably coupled to an assessment platform 204. The measurement device 202 includes a computing device, such as computing device 100. The mobile computing device 206 also includes a computing, such as computing device 100. The assessment platform 204 also includes a computing device, such as computing device 100. It should also be readily understood that computing device 100, measurement device 202, assessment platform 204, and mobile computing device 206, and all of the components thereof, can be physical systems made up of physical devices, cloud-based systems made up of cloud-based devices that store program logic and/or data of cloud-based applications and/or services (e.g., perform at least one function of a software application or an application platform for computing systems and devices detailed herein), or some combination of the two.

An example method for identifying a condition can first involve receiving movement data associated with the movement of a subject over a configurable period of time. The computing system 200 can then associate the received movement data with a profile of the subject. The computing system 200 can then apply a machine learning algorithm to the received movement data and the profiled. By applying the machine learning algorithm, and based, at least in part, on the received movement data and the profile, the computing system 200 can determine a likelihood of a mobility disorder.

As noted above, to determine the likelihood of a mobility disorder, the computing system 200 can first receiving movement data associated with the movement of a subject over a configurable period of time. In examples implementations, the movement data is received by the assessment platform 204.

In example implementations, movement data can include one or more of the following: (i) weight distribution; (ii) balance; (iii) movement patterns; (iv) step distance; (v) step height; (vi) foot contact time; (vii) similarity between legs; (viii) pace; (ix) step cadence; (x) step irregularities; (xi) delay of step on one side; (xii) foot strike; (xiii) heart rate; (xiv) location; and/or (xv) muscle and/or tissue stiffness. Additionally or alternatively, movement data can include a captured image or video of the subject. For instance, the movement data can include a video of the subject walking and/or running. In examples, movement data can also include one or more identifiers of a subject. For instance, the movement data can include the subject's name or another identifier, such as a numeric, alpha, or alphanumeric code associated with the subject. Many examples of movement data are possible.

In examples, a measurement device 202 collects movement data. The measurement device 202 is communicably coupled to the assessment platform 204, for example, according to one or more protocols (e.g., cellular, WI FI, HD-SDI, ethernet cable, etc.). Accordingly, the measurement device 202 is configured to transmit collected movement data to the assessment platform 204. In some examples, the measurement device 202 and the mobile computing device 206 may be the same device (e.g., a smartphone). In some examples, the measurement device 202 is communicably coupled to the mobile computing device 206, for example, according to one or more protocols (e.g., cellular, WI FI, HD-SDI, ethernet cable, etc.). The mobile computing device 206 may be communicably coupled to the assessment platform 204, for example, according to one or more protocols (e.g., cellular, WI FI, HD-SDI, ethernet cable, etc.). In these examples, the measurement device 202 may transmit collected movement data to the mobile computing device 206. The mobile computing device 206 may then transmit the movement data to the assessment platform 204.

An example measurement device 202 can collect data utilizing a variety of different techniques and technologies. In examples, to capture the movement data, the measure device 202 can include an accelerometer and/or a gyroscope to collect movement based data (e.g., weight distribution, balance, movement patterns, step distance, step height, similarity between legs, pace; step cadence, step irregularities, delay of step on one side of the subject, etc.). Additionally or alternatively, the measurement device 202 can include an electrical sensor to measure the heart rate of the subject. Additionally or alternatively, the measurement device 202 can include global positioning system (GPS), radio frequency identification (RFID), or similar, to collect location based data of a subject (e.g., location, pace, movement patterns, etc.). Additionally or alternatively, the measurement device 202 can utilize techniques related to electromyography (EMG), mechano-myography, ultrasound elastography, or similar, to collect data related to muscle and/or tissue stiffness.

In example implementations, the measurement device 202 includes a wearable device, worn by, or otherwise attached to, a subject over a configurable period of time. For instance, example wearable devices can include one or more of the following: (i) one or more boots; (ii) one or more ankle braces; (iii) a harness; (iv) a collar; (v) a tag; (vi) a helmet; and/or (vii) an RFID tag. In example implementations, one or more of these example devices may be attached to a limb (e.g., a leg or ankle) of a subject. Additionally or alternatively, one or more of these example devices may be attached to a neck of a subject. Other examples of wearable devices are possible.

Additionally or alternatively, the measurement device 202 can include a treadmill to collect movement data. For example, a treadmill can include sensors to collect certain movement data such as weight distribution, balance, movement patterns, step distance, step height, foot contact time, similarity between legs, pace, step cadence, step irregularities, delay of step on one side, and/or foot strike. A treadmill can be used a measurement device 202 alone or in combination with other measurement devices (e.g., heart rate monitor and/or one or more boots).

Additionally or alternatively, in some example implementations, the measurement device 202 can include an image capture device and/or sensor, such as a camera on a smartphone. In these examples, the movement data can include one or more pictures or videos of the subject. For instance, in an example implementation, the movement data can include a video of the subject walking or running. Other examples are possible.

In some examples, the measurement device 202 can include a sensor to measure muscle and/or tissue stiffness. For instance, an example measurement device can include a probe that oscillates against a muscle at a certain frequency. The measure device 202 can measure physical displacement of the muscle and/or a response frequency, which can indicate tone, stiffness, and elasticity in the muscle tissue, as well as other biomechanical and elastic properties of the muscle tissue.

The measurement device 202 collects movement data over a configurable period of time. In some examples, the period of time can be relatively short, such as a few second or minutes. For instance, an example measurement device 202, such as a camera on a smart phone, can record a video of a subject walking or running for a few seconds. In other examples, the measurement device 202 can collect data over a period of days, weeks, or months. For instance, a subject may wear a wearable measurement device 202 over a number days. In some examples, the subject may wear a wearable measurement device 202 for a period of time (e.g., one hour) each day over a number of weeks. In another example, movement data measured at different times (e.g., two months apart from each other) can be measured and compared. Many examples are possible.

Once the measurement device 202 collects and transmits movement data to the assessment platform 204, the assessment platform 204 can associate the received movement data with a profile of the subject. In example implementations, the assessment platform 204 can include a database storing a number of profiles associated with different subjects. Profiles can include anatomical data about the subject. For instance, anatomical data can include, but is not limited to, one or more of the following: (i) breed; (ii) sex; (iii) weight; (iv) age; (v) location; and/or (vi) medical record. In some examples, a subject's medical record can include, but is not limited to, subject demographic information, vital signs at each clinical visit, diagnoses, medications, treatment plans, progress notes, subject problems, vaccine history, test results, and imaging data, such as radiographs. The demographic data may include species, breed, weight, age, gender, and geographic location, for example. In some examples, the profile of the subject may also include information on test results (for example, complete blood count (CBC), blood chemistry, pathology, urinalysis, serology, and PCR (polymerase chain reaction) panels/assays), vector of exposure, and diagnoses.

In some example implementations, associating the received movement data with a profile of the subject can include mapping a subject identifier included in the movement data to a subject identifier in the profile (e.g., subject's name and/or numeric, alpha, or alphanumeric code specific to the subject). Other techniques of associating the movement data to profile of a subject are possible.

In some examples, if a profile does not exist, or has not been created for a subject, the assessment platform 204 can create and/or prompt a user (e.g., a pet owner) to create a profile of the subject. For instance, a user may receive a message (e.g., text message, e-mail, notification, etc.) prompting a user to create a profile for a subject. In some examples, this message may be sent to a mobile computing device 206 associated with the subject and/or user.

Once the received movement data is associated with the profile of the subject, the assessment platform 204 can apply a machine learning algorithm to the received movement data and the profile. The assessment platform 204 can then determine via the machine learning algorithm and based at least in part on the received movement data and information obtained from the profile of the subject, a likelihood of a mobility disorder. In some examples, a mobility disorder can include one more of hip dysplasia, osteoarthritis, and/or joint issues. Other example mobility disorders are possible.

In some examples, the machine learning algorithm is used to determine a likelihood of a mobility disorder. As noted above, determining the likelihood of a mobility disorder can be based both on the received movement data and anatomical data (e.g., breed, sex, or weight of the subject). For instance, certain breeds of dogs have an increased likelihood for certain mobility disorders. For example, German Shepherds, Golden Retrievers, Labrador Retrievers, Saint Bernards, Great Danes, and Newfoundlands are more susceptible to hip dysplasia than other breeds. Additionally, female dogs may be more susceptible to hip dysplasia than male dogs. Similarly, larger dogs may be more susceptible to hip dysplasia than smaller dogs.

In an example, the machine learning algorithm may determine there is high likelihood of a mobility disorder if the movement data indicates a severe abnormality in subject's weight distribution between limbs and information in the subject's profile indicates a higher likelihood of a mobility disorder (e.g., the subject is overweight for the particular breed). In another example, the machine learning algorithm may detect a slight limp in the subject's movement data, however information in the subject's profile does not indicate any increased risk factors. In this case, the machine learning algorithm may determine that there is a lower likelihood of a mobility disorder. Many examples are possible.

The machine learning model may be trained using training data that shares a characteristic with a subject to be analyzed by the measurement device 202. Training the machine learning model may include inputting one or more training data samples into the machine learning model, predicting, by the machine learning model, an outcome of a determined condition of the one or more training data samples, comparing the at least one outcome to the characteristic of the one or more training samples, and adjusting, based on the comparison, the machine learning model. For example, if a user is attempting to determine the likelihood of hip dysplasia, the machine learning model may be trained by inputting training data of subjects with known diagnosis of hip dysplasia, predicting, by the machine learning model, a likelihood of hip dysplasia, comparing the predicted likelihood to the known likelihood, and adjusting, based on the comparison, the machine learning model.

In some examples, the training data may include labeled training data (supervised learning), partially labeled training data (semi-supervised learning), or unlabeled training data (unsupervised learning). In some examples, training may include reinforcement learning.

The machine learning model may include an artificial neural network, a support vector machine, a regression tree, an ensemble of regression trees, or some other machine learning model architecture or combination of architectures.

The training data may include data obtained from tests performed either at laboratories or using instruments at the POC terminal, and clinical history data derived from integrated veterinary clinic practice information management software (PIMS). The training data may additionally or alternatively include images or videos of subjects walking or running. In some aspects of the disclosure, the data samples are collected over a period of time and stored in the one or more databases.

In some examples, the machine learning model of the assessment platform 204 may be adjusted based on training such that if the outcome of a determined likelihood matches the likelihood of the training data, the machine learning model is reinforced and if the outcome of a determined likelihood does not match the likelihood of the training data of the training data, the machine learning model is modified. In some examples, modifying the machine learning model includes increasing or decreasing a weight of a factor within the neural network of the machine learning model. In other examples, modifying the machine learning model includes adding or subtracting rules during the training of the machine learning model.

In some examples, determining the likelihood of a mobility disorder can also involve receiving test results data associated with the subject. In examples, the test result data includes data associated with a biological sample of the subject. For instance, in examples, the biological sample of the subject can include, but is not limited to, one or more of the following: blood, urine, saliva, fecal matter, secretion, excretion, Fine Needle Aspirate (FNA), lavage fluids, body cavity fluids, semen, bacteria, ear wax, skin cells, fecal matter, and biopsied samples. Test may additionally include one or more of the following: blood coagulation test, polymerase chain reaction (PCR) test, and/or immunoassay, among other possibilities.

In example implementations, the test result data may include a marker associated with the mobility disorder. For instance, certain levels of glucose, lactate, and pH in synovial fluid (SF) can be used to detect or indicate an increased likelihood of osteoarthritis in dogs. Additionally or alternatively, some pro-inflammatory and degenerative markers, such as, tumor necrosis factor alpha (TNF-alpha), interleukin-1beta (IL-1beta), tenascin-c (TN-C), and matrix metalloproteinase-2 (MMP-2), may be an indicator or osteoarthritis in a subject. Some markers, such as urinary CTX-I and II, serum MMP-9, and serum PIICP, can be used to identify hip dysplasia in subject. In some examples, if a test result reaches a threshold level of a marker, the machine learning algorithm may determine that the subject has a high likelihood of a mobility disorder. In some examples, the determined likelihood

In some example implementations, the assessment platform 204 may change and/or update the machine learning algorithm based at least in part on whether the test result data includes the marker. For instance, in some examples, modifying the machine learning model can includes increasing or decreasing a weight of a factor, such as the existence and/or level of a marker, within the neural network of the machine learning model. In other examples, modifying the machine learning model includes adding or subtracting rules, such as factoring in the existence and/or level of a marker, during the training of the machine learning model.

Once the assessment platform 204 has determined a likelihood of a mobility disorder of one or more subjects, the assessment platform 204 may transmit instructions that cause a computing device (e.g., the computing device 100) to display one or more graphical indications of the determined likelihood. For instance, in some examples, the assessment platform 204 may transmit instructions to a user device, such as a smartphone associated with the subject, to display one or more graphical indications of a determined likelihood that the subject.

In some example implementations, the assessment platform 204 is used to provide alerts to clinicians when a subject has an increased likelihood of a mobility disorder. For instance, further targeted screening may be performed, in response to the alert, to validate the presence/absence of a mobility disorder. This approach provides significant advantages for recognizing and treating mobility disorders by reducing the number of missed diagnoses of early-stage mobility disorders prior to a crisis. Additional potential benefits of this approach include training veterinarians to recognize subjects with mobility disorders prior to the worsening symptoms of the disorder and highlighting the potential significance of nonspecific recurring clinical signs in recognizing the risk for mobility disorders.

Additionally or alternatively, Once the assessment platform 204 has determined a likelihood of a mobility disorder of a subject, the assessment platform 204 may update the profile of the subject with the determination.

### EXAMPLE METHODS AND ASPECTS

Now referring to FIG. 3, an example computer implemented method for identifying a condition. Method 300 shown in FIG. 3 presents an example computer implemented method for identifying a condition that could be used such as the computing device 100 and/or computing system 200 shown in FIGS. 1-2, for example. Further, devices or systems may be used or configured to perform logical functions presented in FIG. 3. In other examples, components of the devices and/or systems may be arranged to be adapted to, capable of, or suited for performing the functions, such as when operated in a specific manner. Method 300 may include one or more operations, functions, or actions as illustrated by one or more of blocks 302-308. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

At block 302, method 300 involves receiving movement data associated with the movement of a subject over a configurable period of time. In some examples, the movement data comprises one or more of: (i) weight distribution; (ii) balance; (iii) movement patterns; (iv) step distance; (v) step height; (vi) foot contact time; (vii) similarity between legs; (viii) pace; (ix) step cadence; (x) step irregularities; (xi) delay of step on one side; (xii) foot strike; (xiii) heart rate; (xiv) location; and/or (xv) muscle and/or tissue stiffness.

At block 304, method 300 involves associating the received movement data with a profile of the subject, the profile comprising anatomical data of the subject. In some examples, receiving the movement data involves receiving the movement data from a wearable device attached to the subject. In some examples, receiving the movement data comprises receiving the movement data from one or more image capture devices. In some examples, receiving the movement data comprises receiving the movement data from one or more accelerometers.

At block 306, method 300 involves applying a machine learning algorithm to the received movement data and the profile.

At block 308, method 300 involves determining, via the machine learning algorithm and based at least in part on the received movement data and the anatomical data, a likelihood of a mobility disorder. In some examples, determining the likelihood of the mobility disorder is based at least in part on a change in the movement data over the configurable period of time.

In some example implementations, method 300 may further involve receiving test results data associated with the subject, the test result data comprising data associated with a biological sample of the subject. Method 300 may also involve determining whether the test result data includes a marker associated with the mobility disorder. Method 300 may additionally involve updating the machine learning algorithm based at least in part on whether the test result data includes the marker.

In some examples, the mobility disorder is osteoarthritis, and the marker is associated with osteoarthritis.

In some examples, the mobility disorder is hip dysplasia, and the marker is associated with hip dysplasia.

The singular forms of the articles “a,” “an,” and “the” include plural references unless the context clearly indicates otherwise. For example, the term “a compound” or “at least one compound” can include a plurality of compounds, including mixtures thereof.

Various aspects and embodiments have been disclosed herein, but other aspects and embodiments will certainly be apparent to those skilled in the art. Additionally, the various aspects and embodiments disclosed herein are provided for explanatory purposes and are not intended to be limiting, with the true scope being indicated by the following claims.

What is claimed is:

1. A computer implemented method for identifying a condition, the method comprising:
receiving data associated with movement of a subject over a configurable period of time;
associating the received movement data with a profile of the subject, the profile comprising anatomical data of the subject;
applying a machine learning algorithm to the received movement data and the profile; and
determining, via the machine learning algorithm and based at least in part on the received movement data and the anatomical data of the subject, a likelihood of a mobility disorder.

2. The computer implemented method of claim 1, further comprising:
receiving test results data associated with the subject, the test result data comprising data associated with a biological sample of the subject;
determining whether the test result data includes a marker associated with the mobility disorder; and
updating the machine learning algorithm based at least in part on whether the test result data includes the marker.

3. The computer implemented method of claim 2, wherein the mobility disorder is osteoarthritis, and the marker is associated with osteoarthritis.

4. The computer implemented method of claim 2, wherein the mobility disorder is hip dysplasia, and the marker is associated with hip dysplasia.

5. The computer implemented method of claim 1, wherein receiving the movement data comprises receiving the movement data from a wearable device attached to the subject.

6. The computer implemented method of claim 1, wherein receiving the movement data comprises receiving the movement data from one or more image capture devices.

7. The computer implemented method of claim 1, wherein receiving the movement data comprises receiving the movement data from one or more accelerometers.

8. The computer implemented method of claim 1, wherein determining the likelihood of the mobility disorder is based at least in part on a change in the movement data over the configurable period of time.

9. The computer implemented method of claim 1, wherein the movement data comprises one or more of: (i) weight distribution; (ii) balance; (iii) movement patterns; (iv) step distance; (v) step height; (vi) foot contact time; (vii) similarity between legs; (viii) pace; (ix) step cadence; (x) step irregularities; (xi) delay of step on one side; (xii) foot strike; (xiii) heart rate; (xiv) location; and/or (xv) muscle and/or tissue stiffness.

10. A non-transitory computer-readable medium, having stored thereon program instructions that, upon execution by a processor, cause performance of a set of operations comprising:
receiving movement data associated with movement of a subject over a configurable period of time;
associating the received movement data with a profile of the subject, the profile comprising anatomical data of the subject;
applying a machine learning algorithm to the received movement data and the profile; and
determining, via the machine learning algorithm and based at least in part on the received movement data and anatomical data of the subject, a likelihood of a mobility disorder.

11. The non-transitory computer-readable medium of claim 10, wherein the set of operations further comprises:
receiving test results data associated with the subject, the test result data comprising data associated with a biological sample of the subject;
determining whether the test result data includes a marker associated with the mobility disorder; and
updating the machine learning algorithm based at least in part on whether the test result data includes the marker.

12. The non-transitory computer-readable medium of claim 11, wherein the mobility disorder is osteoarthritis, and the marker is associated with osteoarthritis.

13. The non-transitory computer-readable medium of claim 11, wherein the mobility disorder is hip dysplasia, and the marker is associated with hip dysplasia.

14. The non-transitory computer-readable medium of claim 10, wherein receiving the movement data comprises receiving the movement data from a wearable device attached to the subject.

15. The non-transitory computer-readable medium of claim 10, wherein receiving the movement data comprises receiving the movement data from one or more image capture devices.

16. The non-transitory computer-readable medium of claim 10, wherein receiving the movement data comprises receiving the movement data from one or more accelerometers.

17. The non-transitory computer-readable medium of claim 10, wherein determining the likelihood of the mobility disorder is based at least in part on a change in the movement data over the configurable period of time.

18. A computing system comprising:
a processor; and
a non-transitory computer-readable medium, having stored thereon program instructions that, upon execution by the processor, cause performance of a set of operations comprising: receiving movement data associated with movement of a subject over a configurable period of time;
associating the received movement data with a profile of the subject, the profile comprising anatomical data of the subject;
applying a machine learning algorithm to the received movement data and the profile; and
determining, via the machine learning algorithm and based at least in part on the received movement data and the anatomical data of the subject, a likelihood of a mobility disorder.

19. The computing system of claim 18, wherein the set of operations further comprises: receiving test results data associated with the subject, the test result data comprising data associated with a biological sample of the subject;
determining whether the test result data includes a marker associated with the mobility disorder; and
updating the machine learning algorithm based at least in part on whether the test result data includes the marker.

20. The computing system of claim 19, wherein the mobility disorder is osteoarthritis, and the marker is associated with osteoarthritis.
